(19) United States
(12) Patent Application Publication
(10) Pub. No.: US 2012/0134985 A1
(43) Pub. Date: May 31, 2012

(54) METHODS FOR MODULATING METABOLIC AND CIRCADIAN RHYTHMS

(75) Inventors: Ronald Evans, LaJolla, CA (US); Katja A. Lamia, San Diego, CA (US); Reuben J. Shaw, San Diego, CA (US)

(73) Assignee: The Salk Institute for Biological Studies, LaJolla, CA (US)

(21) Appl. No.: 13/257,758

(22) PCT Filed: Mar. 22, 2010
(86) PCT No.: PCT/US2010/028196

§ 371 Date: Jan. 27, 2012

Related U.S. Application Data
(60) Provisional application No. 61/162,219, filed on Mar. 20, 2009.

(51) Int. Cl. A61K 39/395; A61P 25/00; C07K 16/42; A61P 3/00; G01N 33/566; C12Q 1/48

(52) U.S. Cl. 424/131.1; 436/501; 435/15; 530/387.2

(57) ABSTRACT

The role of AMPK in arcadian rhythms and methods of screening for agents that modulate such rhythms are disclosed. Compositions that are useful for modulating such rhythms and uses thereof are also disclosed.

### CROSS-REFERENCE TO RELATED APPLICATIONS

This application claims the benefit of U.S. Provisional Application No. 61/162,219, filed Mar. 20, 2009, herein incorporated by reference.

### ACKNOWLEDGEMENT OF GOVERNMENT SUPPORT

This work was supported by National Institutes of Health Grant Nos. DK057978, DK062434, CA104838, DK080425, and EY016807. The Government of the United States has certain rights in this invention.

### FIELD OF THE INVENTION

This disclosure concerns the use of agonists and antagonists of AMP-activated protein kinase (AMPK) for modulating circadian rhythms. More particularly, the disclosure provides compositions and methods for screening and modulating sleep behavior.

### BACKGROUND

Circadian clocks coordinate behavioral and physiological processes with daily light-dark cycles by driving rhythmic transcription of thousands of genes in mammalian tissues.

### SUMMARY

The disclosure demonstrates that AMPK phosphorylates the transcription repressor CRY1 and CRY2 and stimulates their proteasomal degradation. Furthermore the disclosure demonstrates that cryptochromes bind and regulate the transcriptional activity of several nuclear hormone receptors in addition to their established function in mammalian circadian clocks. The disclosure also demonstrates that cryptochrome proteins are required for a subset of the transcription responses to treatment with AMPK-activating drugs. Accordingly, the pharmacological modulation of cryptochromes will be useful in the treatment of metabolic disorders.

The use of small molecule drugs that modulate cryptochrome transcriptional co-regulator function will be useful in the treatment of metabolic disorders due to the demonstration the cryptochromes regulate the transcriptional activity of established metabolically important transcription factors including, but not limited to, the peroxisome proliferator activated receptors PPAR alpha, beta, delta and gamma. Because cryptochromes bind and are regulated by natural small molecules co-factors (the catalytic cofactor flavin adenine dinucleotide or FAD and a light harvesting cofactor 5,10-methenyl tetrahydrofolyl polyglutamate or MTHF), the cryptochromes are good targets for regulation by the synthetic small molecules.

The disclosure demonstrates that the energy sensor AMPK modifies two serines in CRY1, whose phosphorylations mediate CRY1-FBXL3 interaction and the proteasomal degradation of CRY1. Thus, while CRY1 originally evolved as a photoreceptor, posttranslational modification could endow it as a key signaling mediator. Genetic or pharmacological manipulation of AMPK in vivo alters both cryptochrome stability and circadian rhythms, suggesting a novel entrainment mechanism by which nutrient-regulated signals are able to reset circadian clocks in mammalian peripheral organs.

The disclosure provides methods and compositions for modifying circadian rhythms in a mammalian subject such as a human. The disclosure demonstrates that AMPK is modified during the circadian cycle of mammalian subjects both in the brain and in other tissues in the body. In one embodiment, the disclosure provides the use of an AMP kinase agonist or antagonist for the manufacture of a medicament to modulate circadian rhythms in a subject. In one embodiment, the AMPK agonist is AICAR. In another embodiment, the AMPK antagonist is an antibody or a compound C or analog or derivative thereof. In yet another embodiment, the AMPK agonist comprises a formulation or derivation that is capable of crossing the blood brain barrier. In yet a further embodiment, the AMPK agonist is formulated for oral administration, intravenous injection, intramuscular injection, epidural delivery, intracranial or subcutaneous injection.

The disclosure also provides a composition comprising an AMPK agonist formulated in combination with a second active ingredient that modifies circadian rhythms. In one embodiment, the second active ingredient is a sleep aid. In a further embodiment, the composition is formulated for oral administration, intravenous injection, intramuscular injection, epidural delivery, intracranial delivery, or subcutaneous injection.

The disclosure provides a method for modulating sleep in a mammal comprising, administering to the mammal an effective amount of an AMPK agonist or antagonist to modulate circadian rhythms in a mammal.

The disclosure also provides a method for identifying an agent that modulates circadian rhythms or sleep in a subject, comprising: (a) contacting a sample comprising a AMPK pathway with at least one test agent; and (b) comparing an activity of the AMPK or AMPK pathway in the presence and absence of the test agent wherein a test agent the changes that activity is indicative of an agent that circadian rhythm modulating activity.

The disclosure also provides a method of identifying an agent for use in modulating metabolism or circadian rhythms, comprising contacting the agent with a Cry1 or Cry2 protein and measuring the ability of the agent to phosphorylate or dephosphorylate a Cry1 or Cry2 or modify the stability or expression of Cry1 or Cry2, wherein an agent the modifies Cry1 or Cry2 is an agent useful for modulating metabolism or circadian rhythms. In one embodiment, the agent decreases the stability of Cry1 or Cry2.

The disclosure also provides a composition comprising an agent identified by the method above, wherein the agent decreases the stability of Cry1 or Cry2.

The disclosure also provides a method of treating a metabolic or circadian disease or disorder comprising contacting the subject with an agent or composition of the disclosure wherein the agent or composition promotes the phosphorylation or dephosphorylation of Cry1 and/or Cry2. In one embodiment, the agent or composition modulates cryptochrome transcriptional co-regulator function. In another embodiment, the agent or composition modulates the peroxisome proliferator activated receptors (PPAR) alpha, beta (delta) and gamma. In yet another embodiment, the agent is an AMPK agonist selected from the group consisting of biguanide derivatives, AICAR, metformin or derivatives thereof, phenformin or derivatives thereof, leptin, adiponectin, AICAR (5-aminoimidazole-4-carboxamide, ZMP, DRL-16536, BG800 compounds (Betagenon), and furan-2-carboxylic acid derivative.

The disclosure also provides a method of determining a metabolic or circadian rhythm disease or disorder comprising measuring the stability of CRY1 or CRY2 in a tissue during a 24 hour period, wherein a period of long-term stability of CRY1 or CRY2 in the presence normal or excess ATP concentrations is indicative of a metabolic or circadian rhythm disease or disorder. In one embodiment, the method utilizes an antibody that specifically binds to an epitope comprising S71 or S280 of mCRY1.

The disclosure also provides a method of promoting rest and fat catabolism comprising administering an AMPK agonist during a nocturnal phase of a circadian cycle, wherein the AMPK agonist decreases the stability of CRY1 or CRY2.

The disclosure also provides a method of treating a metabolic or circadian rhythm disorder comprising administering an AMPK agonist during a rest period of a circadian cycle.

The foregoing and other features will become more apparent from the following detailed description of several embodiments, which proceeds with reference to the accompanying figures.

### BRIEF DESCRIPTION OF THE FIGURES

FIG. 1A-E shows phosphorylation of S71 or S280 destabilizes mCRY1 by altering interactions with FBXL3 and PER2. (A) AD293 cells expressing Flag-tagged mCRY1 with the indicated mutations were treated with 100 μg/ml cycloheximide (CHX) for the indicated times. Flag-mCRY1 was detected by western blotting. Immunoblot for β-actin was used as a loading control. (B) AD293 cells expressing CLOCK, BMAL1, Pert-luciferase and the indicated amounts and alleles of mCRY1 were examined for luciferase activity 48 hrs after transfection. (C) Flag-mCRY1 was immunoprecipitated from AD293 cells transiently expressing the indicated plasmids. FBXL3 bound to CRY1 was detected by immunoblotting for the v5 epitope tag. (D) AD293 cells transiently expressing CLOCK, BMAL1, Pert-luciferase and the indicated alleles of mCRY1 with or without co-expression of FBXL3 as indicated were examined for luciferase activity 48 hrs after transfection. ** p<0.01 relative to AA; ## p<0.01 relative to equivalent samples not expressing FBXL3. (E) Flag-mCRY1 was immunoprecipitated from AD293 cells transiently expressing the indicated alleles of CRY1 with or without co-expression of PER2. PER2 bound to CRY1 was detected by immunoblotting.

FIG. 2A-G shows AMPK destabilizes mCRY1 via Ser71, Ser280 phosphorylation. (A) Sequence alignments showing evolutionary conservation of the regions surrounding S71 of mCRY1 in cryptochrome circadian transcriptional repressors (species names in red font) and blue light photoreceptors (species names in blue font). The highlighted numbers above the sequences indicate amino acid preferences at those positions relative to the target serine for phosphorylation by AMP kinase: red indicates a preference for acidic residues (K/R) and green for hydrophobic residues (L/I/V/F). (B) Top: sequence alignment of the phospho-peptides against which antibodies to mCRY1-pS71 and mACC1-pS79 were raised. Bottom: Both anti-mCRY1-pS71 and anti-ACC1-pS79 antibodies recognize WT but not S71A Flag-mCRY1 immunoprecipitated from AD293 cells. (C) anti-mCRY1-p571 was used to detect phosphorylation of Ser71 in Flag-CRY1 immunoprecipitated from AD293 cells transiently expressing wild type CRY1 (WT) or CRY1S71A (S71A) in the absence or presence of activated alleles of AMPKα1 (CAα1) or AMPKα2 (CAα2). Transiently expressed myc-CAα1 and myc-CAα2 were immunostained with polyclonal rabbit antibodies raised against the myc epitope tags and anti-rabbit AF488 (green). Nuclei were counterstained with DAPI (blue). (D) HeLa cells transiently expressing Flag-CRY1 with wild type (WT) or kinase dead (KD) LKB1 were treated with vehicle (−) or 2 mM AICAR (+) for 2 hours. (E) AD293 cells transiently expressing the indicated alleles of Flag-mCRY1 were treated with media containing 25 mM or 0.5 mM glucose. (F) Paired wild type (AMPK+/+) and ampkα1^{-/-};ampkα2^{-/-} (AMPK−/−) mouse embryonic fibroblasts stably expressing Flag-tagged wild type CRY1 (WT) or CRY1S71A/S280A (AA) were treated with vehicle (−) or 2 mM AICAR (+) for 2 hours. (G) MEFs described in (F) were treated with 100 μg/ml cycloheximide (CHX) for the indicated times. CRY1 was detected by immunoprecipitation and immunoblotting for the Flag epitope in (D-G).

FIG. 3A-D shows disruption of AMPK signaling alters circadian rhythms in MEFs. (A) Unsynchronized paired wild type (AMPK^{+/+}) or ampkα1^{-/-}; ampkα2^{-/-} (AMPK−/−) mouse embryonic fibroblasts were stimulated by 2 hour exposure to 50% horse serum followed by transfer to media containing 25 mM glucose, 0.5 mM glucose or 25 mM glucose supplemented with 1 mM AICAR. Quantitative PCR analysis was performed using cDNA samples collected at the indicated times following stimulation. Data represent the mean of two independent experiments, each analyzed in triplicate. (B) Fibroblasts stably expressing Bmall-luciferase were cultured in media containing the indicated amounts of glucose with or without 2 mM AICAR. Typical results of continuous monitoring of luciferase activity are shown. (C and D) Quantitation of the circadian period (C) and amplitude (D) of Bmall-driven luciferase activity from experiments performed as described in (B). Data in (C) and (D) represent the mean±standard deviation for four samples per condition. ANOVA analysis indicated a significant difference between categories. ** P<0.01 vs. samples cultured in 25 mM glucose in Scheffe's post-hoc analysis.

FIG. 4A-C shows AMPK activity and nuclear localization undergo circadian regulation. (A) Immunoblotting for phospho-Raptor-S792 (pRaptor), Raptor, phospho-ACC1-S79 (pACC1) and ACC1 were performed in whole cell lysates prepared from mouse livers collected at the indicated circadian times. The blots are representative of three independent experiments. (B) Quantitative PCR analysis of cDNA prepared from mouse livers collected at the indicated circadian times. Each data point represents the mean±standard deviation of three samples each taken from a unique animal and analyzed in quadruplicate. (C) Nuclear extracts were prepared from the livers of two mice at each of the indicated circadian times. Protein levels of AMPKα1, AMPKα2, PER2, CRY1 and REVERBα were analyzed by immunoblotting. Nuclear extracts from paired wild type (α1+/+) and ampkα1^{-/-} (α1−/−) or wild type (α2+/+) and ampkα2^{-/-} (α2−/−) mice collected at the indicated circadian times were used as controls for antibody specificity.

FIG. 5A-C shows AMPK activation alters CRY stability and circadian rhythms in mouse livers. (A) Mice were injected with saline or 500 mg AICAR per kg of bodyweight and liver samples were collected one hour later at zeitgeber time (ZT, hours after lights on) 6 or ZT18. Endogenous CRY1 was detected by immunoblotting in liver nuclear extracts. n.s. denotes a non-specific band to assess sample load. Samples collected from wild type (CRY+/+) and cry1^{-/-};cry2^{-/-} (CRY−/−) mice were used as controls for antibody specificity. Data represents a typical result from two independent experiments. (B) LKB1^{+/+} and LKB1^{fl/fl} mice were injected with adenovirus expressing Cre recombinase (Ad-Cre) via the tail vein. One to two weeks after Ad-Cre injection, mice were transferred to constant darkness and livers were collected at the indicated circadian times. CRY1, PER2, and REVERBa, were detected by immunoblotting. (C) cDNA samples prepared from the livers described in (B) were analyzed by quantitative PCR analysis of dbp, reverba, cry1, and per2 expression. All transcripts were normalized to u36b4 as an internal control. Each data point represents the mean±standard deviation of three samples analyzed in quadruplicate.

FIG. 6 shows AMPK contributes to metabolic entrainment of peripheral clocks. Model depicting the role of AMPK in metabolic entrainment of peripheral circadian clocks in mice: During the day, nuclear localization of AMPK increases in concert with its probable activation by reduced dietary and circulating glucose. Active nuclear AMPK phosphorylates cryptochromes, thus increasing their interaction with FBXL3 and leading to proteasomal degradation, resulting in the activation of clock-controlled genes (ccg's). At night, reduced nuclear AMPK activity allows nuclear accumulation of cryptochromes and repression of ccg's.

FIG. 7A-D shows the identification of mCRY1 phosphorylation sites. (A) Flag-mCRY1 purified from transiently transfected AD293 cells was analyzed by LC-MS/MS for the presence of phosphorylated serine, threonine and tyrosine residues. Kinases predicted to catalyze the observed phosphorylations were predicted using a combination of literature searches and the Scansite program (http:(//)scansite.mit.edu). Sequence conservation was determined using MegAlign. (B) The thickness of the orange bars below the schematic diagram of the CRY1 protein indicates the relative number of peptides observed by LCMS/MS for each region of the protein sequence. (C) Phosphorylation sites predicted by Scansite that may not be observable in our LC-MS/MS analysis based on the peptide coverage shown in B. (D) Flag-mCRY1 with the indicated mutations was expressed in AD293 cells which were treated with 100 μg/ml cycloheximide for the indicated times. CRY1 proteins were detected by immunoblot for the Flag tag.

FIG. 8 shows mCRY1 5280 sequence conservation. mCRY1 S280 is surrounded by a conserved AMPK substrate motif: Sequence alignments showing evolutionary conservation of the regions surrounding 5280 of mCRY1 in cryptochrome circadian transcriptional repressors (species names in red font) and blue light photoreceptors (species names in blue font). The highlighted numbers above the sequences indicate amino acid preferences at those positions relative to the target serine for phosphorylation by AMP kinase: red indicates a preference for acidic residues (K/R) and green for hydrophobic residues (L/I/V/F) at the indicated positions.

FIG. 9 shows purified AMPK phosphorylates mCRY1 in vitro. Flag-tagged mCRY1 purified from AD293 cells was incubated for 30 minutes with #³²P-ATP in the absence or presence of AMP kinase and 300 μM AMP as indicated. Phosphorylation of mCRY1 was detected by autoradiography; total mCRY1 levels were determined by immunoblot for the Flag epitope. Purified AMPK efficiently phosphorylated purified mCRY1 and this phosphorylation was strongly activated by the presence of AMP, confirming that the relevant kinase in the purification mixture is AMPK and not another associated kinase.

FIG. 10A-D show disruption of AMPK alters circadian rhythms in MEFs. 3T3 immortalized mouse embryonic fibroblasts (A) or paired wild type (AMPK^{+/+}) or ampkα1^{-/-};ampkα2^{-/-} (AMPK^{-/-}) fibroblasts (B) were stimulated by 2 hour exposure to 50% horse serum followed by transfer to media containing 25 mM glucose (black symbols), 0.5 mM glucose (gray symbols) or 25 mM glucose supplemented with 1 mM AICAR (red symbols). Quantitative PCR analysis was performed using cDNA samples prepared from lysates collected at the indicated times following stimulation. Data represent the mean±standard deviation of two or three independent experiments each analyzed in triplicate.

FIG. 11 shows mCRY1 interacts with nuclear hormone receptors. AD293 cells co-expressing Flag-tagged mCRY1 with various v5-tagged nuclear hormone receptors were lysed and protein complexes containing mCRY1 were isolated by immunoprecipitation of the Flag tag. The presence of individual nuclear hormone receptors in the mCRY1-containing protein complexes was detected by immunoblot for the v5 tag (top). The amount of mCRY1 in the immunoprecipitated complexes is shown by immunoblot for the Flag tag (middle). The amount of each nuclear hormone receptor present in the lysates is shown by immunoblot of the v5 tag in a sample taken from the lysates prior to immunoprecipitation (bottom). RORa,b,g (retinoic acid receptor related orphan receptor a, b, g), RXRa,b (retinoid X receptor a, b), PPARd,g (peroxisome proliferator activated receptor d,g), VDR (vitamin D receptor), PXR (pregnane X receptor), CAR (constitutive androstane receptor), ERb (estrogen receptor b), ERRa,b,g (estrogen related receptor a,b,g), GR (glucocortiocoid receptor), MR (mineralocorticoid receptor), PR (progesterone receptor), AR (androgen receptor). Data represent a typical result of two or three independent experiments.

FIG. 12 shows cryptochromes are required for some transcriptional responses to AMPK activation. Wildtype (WT) or Cry1^{-/-};Cry2^{-/-} (CRY^{-/-}) mice were injected with either saline (black bars) or 500 mg AICAR per kg of bodyweight (red bars) at 6:00 pm. cDNA was prepared from livers collected four hours later at 10:00 pm and gene expression was analyzed by quantitative PCR using Sybr GreenER chemistry. Fas (fatty acid synthase) is shown as an example of a gene that is activated by AICAR regardless of Cry1 and Cry2 genotype. Por (p450 oxidoreductase) is shown as an example of a gene whose AICAR-induced activation requires cryptochromes. Data represent the mean±s.e.m. for 3-5 mice per condition.

FIG. 13 show Loss of cryptochromes alters metabolic function in mice. 10-week-old male wildtype (WT, black bars) and Cry1^{-/-}; Cry2^{-/-} (CRY^{-/-}, grey bars) mice were weighed and their resting blood glucose was measured by tail vein nick at 1:00 pm. Data represent the mean±s.e.m. for 10 animals per genotype.

### DETAILED DESCRIPTION

Unless specifically noted otherwise herein, the definitions of the terms used are standard definitions used in the art of pharmaceutical sciences. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the disclosure. Nothing herein is to be construed as an admission that the inventors are not entitled to antedate such disclosure by virtue of prior disclosure.

Circadian rhythms optimize biological efficiency by coordinating appropriate timing of physiological, endocrine and behavioural processes, such as, without limitation, modulation of sleep cycles, energy modulation associated with exercise and calorie reduction, and feeding/nourishment behaviours. Circadian rhythms are thought to contain at least three elements: (a) input pathways(s) that relay environmental information to a circadian pacemaker (clock); (b) a circadian pacemaker that generates the oscillation; and (c) output pathway(s) through which the pacemaker regulates various output rhythms.

The mammalian hypothalamic suprachiasmatic nucleus (SCN) acts as a master pacemaker aligning behavioral and physiological rhythms to light-dark cycles. Initially, the SCN was thought to be the only site of self-sustaining molecular pacemakers in mammals but multiple reports have subsequently shown that such molecular clocks are nearly ubiquitous. Unlike the SCN clock, circadian clocks in non-light sensitive peripheral organs are entrained by daily rhythms of feeding, theoretically allowing peripheral tissues to anticipate daily food consumption and to optimize the timing of metabolic processes. A number of reports support roles for mammalian circadian clocks in regulating the transcription of key metabolic enzymes and in metabolic physiology.

As used herein, the term “circadian rhythm” is intended to mean the regular variation in physiologic and behavioral parameters that occur over the course of about 24 hours. Such activities include the sleep cycle and nourishment cycle, as well as others.

As used herein, the term “modulating” when used in reference to circadian rhythm is intended to mean altering a physiological function, endocrine function or behavior that is regulated by the circadian timing system of an animal, or altering a cellular function that exhibits circadian rhythmicity. Exemplary physiological functions regulated by the circadian timing system of an animal include body temperature, autonomic regulation, metabolism, and sleep-wake cycles. Exemplary metabolic functions include control of weight gain and loss, including increase or decrease in body weight and increase or decrease in percent body fat, modifying endurance behavior, weight loss and the like. Exemplary endocrine functions regulated by the circadian timing system of an animal include pineal melatonin secretion, ACTH-cortisol secretion, thyroid stimulating hormone secretion, growth hormone secretion, neuropeptide Y secretion, serotonin secretion, insulin-like growth factor type I secretion, adrenocorticotropic hormone secretion, prolactin secretion, gamma-aminobutyric acid secretion and catecholamine secretion. Exemplary behaviors regulated by the circadian timing system of an animal include movement (locomotor rhythm), mental alertness, memory, sensorimotor integration, feeding, REM sleep, NREM sleep and emotion.

The AMP-activated protein kinase (AMPK) has been recognized as a central mediator of metabolic signals that is well conserved throughout phylogeny. AMPK is a heterotrimeric protein kinase comprising a catalytic (α) subunit and two regulatory (β,γ) subunits. It is activated when it is phosphorylated by LKB1 in the presence of high AMP/ATP ratios or by CAMKK3 in the presence of elevated intracellular calcium. Biochemical and bioinformatic studies have established the optimal amino acid sequence context in which phosphorylation by AMPK is likely.

AMP-activated protein kinase (AMPK) and AMPK kinase (AMPKK) are associated with a protein kinase cascade. The AMPK cascade regulates fuel production and utilization intracellularly. For example, low cellular fuel (e.g., an increase in AMP concentration) increase AMPK activity. Once activated, AMPK functions either to conserve ATP or to promote alternative methods of ATP generation. Thus, modulating its activity can increase catabolism of energy stores, reducing fat content to increase ATP, or place the body in a resting state to conserve ATP use.

AMPK is expressed in a number of tissues, including the liver, brain, and skeletal muscle. Activation of AMPK has been shown to activate hepatic fatty acid oxidation and ketogenesis, inhibit cholesterol synthesis, lipogenesis, and triglyceride synthesis, inhibit adipocyte lipolysis and lipogenesis, stimulate skeletal muscle fatty acid oxidation and muscle glucose uptake, and modulate insulin secretion by pancreatic beta-cells.

Triggering the activation of AMPK can be carried out with increasing concentrations of AMP. The γ subunit of AMPK undergoes a conformational change so as to expose the active site (Thr-172) on the α subunit. The conformational change of the γ subunit of AMPK can be accomplished under increased concentrations of AMP. Increased concentrations of AMP will give rise to the conformational change on the γ subunit of AMPK as two AMPs bind the two Bateman domains located on that subunit. This role of AMP is demonstrated in experiments that show AMPK activation via an AMP analogue 5-amino-4-imidazolecarboxamide ribotide (ZMP) which is derived from 5-amino-4-imidazolecarboxamide riboside (AICAR). Similarly, antagonists of AMP include the use of inhibitory antibodies that inhibit the activation of downstream kinases by AMPK.

Sleep deprivation (SD) increases neuronal activity. Sustained neuronal activity decreases the cellular energy charge (AMP levels increase and ATP decrease). This in-turn causes a change in the cellular energy sensor AMPK. AMPK, as discussed above, modulates various kinase cascades, including cascades that lead to conservation of ATP.

CLOCK and BMAL1 are polypeptides that upon forming a heterodimer induce transcription of genes associated with circadian rhythms. During a typical circadian cycle, molecular mechanism oscillate between two cycles forming an internal clock having two interconnected transcription/translation feedback loops. The positive arm of the feedback loop is driven by a basic helix-loop-helix-PAS (Per-Arnt-Sim) domain-containing transcription factors CLOCK and BMAL1. The CLOCK/BMAL1 heterodimer activates transcription of the clock genes cryptochrome (Cry1 and Cry2), period (Per1 and Per2), and Rev-Erbα. PER and CRY proteins translocate to the nucleus, where they interact with CLOCK/BMAL1 to down-regulate transcription, generating the negative arm of the major feedback loop.

Robust oscillations of the aforementioned circadian transcriptional program require posttranslational modifications of core clock proteins. Three studies recently identified the F-box protein FBXL3 as a mediator of cryptochrome ubiquitination and degradation. The binding of F-box proteins to their cognate substrates is often regulated by phosphorylation of one or more amino acids within the substrate protein but no such regulatory modification was described for the CRY:FBXL3 interaction.

Posttranslational modification of clock proteins (e.g., phosphorylation and dephosphorylation) determines the protein's localization, intermolecular interactions, and stability and thus regulates the period of the circadian clock. The disclosure demonstrates that this posttranslational regulation can be modulated by AMPK activity and thus AMPK agonist and antagonist can play a role in regulating circadian clock.

Cryptochromes (Cry1 and Cry2) function as circadian photoreceptors in most plants. Cryptochromes are found to be expressed in all tissues; however, expression is higher in the retina and restricted to the inner retina in both mice and humans. In the brain, Cry1 is expressed in the SCN, and expression exhibits a daily oscillation, peaking at about 2:00 p.m. and reaching its lowest at around 2:00 a.m.

Both human cryptochromes have been purified from HeLa cells expressing the Cry genes ectopically and from E. coli as recombinant proteins. Proteins isolated from both sources contain FAD and a pterin.

While cryptochrome evolved as a light sensor, it has been retained as a critical component of the core circadian clock, even in non-light sensitive tissue. The disclosure demonstrates that cryptochromes have been repurposed by AMPK to transduce nutrient signals to the clock. Evidence for reciprocal regulation between circadian and metabolic systems has been mounting over the last decade and an emerging theory suggests that circadian clocks enable the temporal segregation of metabolic processes. While metabolic signals have been shown to set the timing of circadian clocks in mammalian peripheral organs, the molecular mechanisms that transmit such signals have remained unclear.

The disclosure demonstrates that the phosphorylation of cryptochromes by AMPK promotes degradation by association with FBXL3, relieving CLOCK:BMAL1 repression. This process is suppressed by excess glucose and enhanced by AMPK activators such as AICAR and by the nuclear translocation of the ampkP2 regulatory subunit. Accordingly, the disclosure provides a novel biochemical route by which the status of intracellular bioenergetics can directly impact circadian clocks in peripheral tissues.

The circadian activation of AMPK contributes to the maintenance of rhythms by driving the phosphorylation of CRY1 and stimulating its FBXL3-mediated degradation. AMPK phosphorylates CRY1 on two serine residues (S71 and S280 in mouse CRY1). Serine 71 and the surrounding sequence is present in all light-independent cryptochrome transcriptional repressors suggesting that this pathway evolved to enable the metabolic entrainment of circadian clocks that are not exposed to light.

AMPK activity can be regulated by glucose availability in an LKB1-dependent manner and changes in nutrient availability or AMPK activity alter the amplitude and period of the clock in cultured fibroblasts. In vivo, the AMPK substrates ACC1 and Raptor exhibit circadian changes in phosphorylation, suggesting that cytoplasmic and nuclear pathways downstream of AMPK are rhythmically regulated. Given that AMPK is a central regulator of metabolic processes, this has profound implications for the circadian regulation of metabolism. Genetic alteration of circadian clock function either ubiquitously or in a tissue-specific manner elicits changes in feeding behavior, body weight, running endurance and glucose homeostasis, each of which is also altered by manipulation of AMPK. Collectively, these data support the idea that AMPK may be an important mediator of circadian physiological regulation both at the cellular level and at the level of the whole organism.

Interestingly, the transcription, nuclear localization and activation of distinct AMPK subunits exhibit circadian rhythms in mouse hepatocytes, peaking at the time of minimal cryptochrome protein abundance. ampkβ2 transcription is robustly circadian, 8-fold higher in the middle of the day than at night. AMPKβ2 drives the nuclear localization of AMPK and correspondingly rhythmic nuclear accumulation of AMPKα1. Thus, AMPK subunits not only contribute to the regulation of circadian clocks but are themselves transcriptionally regulated in a circadian fashion.

The communication of nutritional status to clocks is complex and that additional pathways contribute to their entrainment in vivo. Two recent studies demonstrated that SIRT1 is rhythmically expressed in hepatocytes and contributes to circadian rhythmicity in fibroblasts. SIRT1 likely plays a role in the metabolic entrainment of circadian clocks due to regulation of its deacetylase activity by NAD+/NADH ratios. Multiple reports suggest a role for heme in the regulation of various clock components and suggest that differential regulation by ferric and ferrous heme transmits information about cellular redox status to circadian clocks. One or more of these mechanisms, and/or diurnal humoral signals or neuronal signals emanating from the SCN probably contributes to the residual circadian rhythms that were observed in the livers of LKB1^{L/L} mice.

Mutations in Fbx13 or Lkb1 are prevalent in human tumors. The demonstration that the LKB1- and AMPK-mediated phosphorylation of cryptochromes stimulates their FBXL3-mediated degradation indicates that two tumor suppressors cooperate in the destabilization of cryptochromes, suggesting that aberrantly high levels of cryptochromes may contribute to cell cycle deregulation or tumorigenesis. In a report describing circadian regulation of liver regeneration, Matsuo and coworkers showed that the livers of cry1^{-/-};cry2^{-/-} mice regenerated more slowly than those of wild type littermates, supporting the idea that CRY proteins play a stimulatory role in cell growth or proliferation. The identification herein of the LKB1- and AMPK-dependent phosphorylation sites that mediate CRY:FBXL3 interaction clarify these questions.

While other phosphorylation sites in mammalian cryptochromes may mediate additional input signals to circadian clocks, the disclosure demonstrates that AMPK-mediated phosphorylation of serines 71 and 280 stimulates CRY1 proteasomal degradation by increasing its interaction with FBXL3. Furthermore, glucose deprivation decreases cryptochrome stability, alters circadian transcripts, and increases circadian period length in cultured cells and that these effects are mediated by AMPK. Furthermore, the genetic disruption of AMPK in mice disrupts cryptochrome stability and circadian rhythms. Together, these data demonstrate that cryptochrome phosphorylation by AMPK has evolved to allow entrainment of peripheral organ clocks by metabolic signals.

The disclosure provide the use of compounds that bind to or otherwise activate or inactivate the AMP-activated protein kinase (AMPK), some of which are currently used for the treatment of diabetes, to influence sleep or other circadian processes. The disclosure demonstrates that genetic or pharmacological manipulation of AMP-activated protein kinase activity alters circadian rhythms in cultured cells and in the livers of intact animals. The disclosure also demonstrates that AMP kinase is expressed in the suprachiasmatic nucleus (SCN), the location of the so-called “master pacemaker” that governs the timing of sleep-wake cycles and other physiological rhythms. Currently available therapies do not cross the blood brain barrier and would therefore not be useful for the modulation of sleep disorders.

The regulation of circadian rhythms by AMPK suggest that AMPK modulators that cross the blood brain barrier would be useful in the treatment of sleep disorders including, but not limited to, insomnia by regulating downstream kinase activity associated with circadian rhythms. In addition, certain circadian polypeptides including, but not limited to, CLOCK, BMAL1, PER and CRY-1 and -2 are regulated by phosphorylation and dephosphorylation and are present in tissues outside the brain. Accordingly, modulating AMPK activity in non-neurological tissue may also be important for setting a circadian rhythm through the kinase cascade and ultimately the regulation of downstream polypeptide phosphorylation and dephosphorylation.

Furthermore, the disclosure demonstrates that the phosphorylation and dephosphorylation of Cry1 and Cry2 have circadian effects and thus are useful targets for modulating a sleep state and energy metabolism. For example, specifically modulating the phosphorylation or dephosphorylation of serines 71 and 280 of CRY1 can promote proteasomal degradation by increasing its interaction with FBXL3.

A number of pharmacological agents that activate AMPK are currently in clinical use for the treatment of diabetes and are in clinical trials for some types of cancer.

AMP kinase agonists such as AICAR have been studied for insulin regulation, diabetes and obesity. However, AMP kinases have not previously been demonstrated to modulate circadian rhythms or sleep behavior. The disclosure demonstrates that modulating AMPK activity can have an effect on downstream processes including the posttranslational modification of proteins associated with circadian rhythms. In one embodiment, the disclosure provides that AMPK agonists and antagonists can be used to modulate circadian rhythm in a subject. For example, AMPK is demonstrated by the disclosure to play a role in the modulation of the transcription activating heterodimer CLOCK/BMAL1.

Various AMPK agonist are known in the art. Methods and compositions comprising such AMPK agonist are provided herein. The use of such AMPK agonist can provide methods for modulating circadian rhythms. Various AMPK agonists are described herein and are known in the art. In one embodiment, the AMPK agonist comprises an AICAR compound. Other compounds useful in the method of the disclosure include biguanide derivatives, analogs of AICAR (such as those disclosed in U.S. Pat. No. 5,777,100, hereby incorporated by reference herein) and prodrugs or precursors of AICAR (such as those disclosed in U.S. Pat. No. 5,082,829, hereby incorporated by reference herein), which increase the bioavailability of AICAR, all of which are well-known to those of ordinary skill in the art. Other activators of AMPK include those described in U.S. Patent Publication No. 20060287356 to Iyengar et al. (the disclosure of which is incorporated herein by reference). Conventionally known AMPK-activating compounds include, in addition to the aforementioned leptin, adiponectin, and metformin, AICAR (5-aminoimidazole-4-carboxamide). Other AMPK agonists include, but are not limited to, DRL-16536 (Dr. Reddy's/Perlecan Pharma), BG800 compounds (Betagenon), furan-2-carboxylic acid derivative (Hanall, K R; see also Int'l. Application Publ. WO/2008/016278, incorporated herein by reference), A-769662 (Abbott) (structure I; see also, Cool et al., Cell Metabol. 3:403-416, 2006); AMPK agonist under development by Metabasis as set forth in Int'l. Publication No. WO/2006/033709; MT-39 series of compounds (Mercury Therapeutics); and AMPK agonist under development by TransTech Pharma.

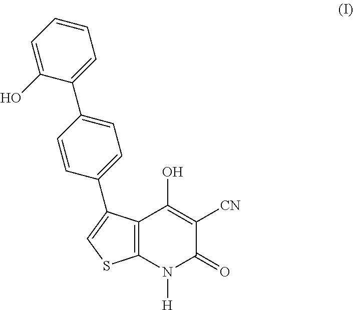

AICAR, for example, is taken into the cell and converted to ZMP, an AMP analog that has been shown to activate AMPK. ZMP acts as an intracellular AMP mimic, and, when accumulated to high enough levels, is able to stimulate AMPK activity (Corton, J. M. et. al. Eur. J. Biochem. 229: 558 (1995)). However, ZMP also acts as an AMP mimic in the regulation of other enzymes, and is therefore not a specific AMPK activator (Musi, N. and Goodyear, L. J. Current Drug Targets—Immune, Endocrine and Metabolic Disorders 2:119 (2002)).

The disclosure provides methods for stimulating a particular cycle of the circadian clock in a subject by either using an AMPK agonist or AMPK antagonist. In one embodiment, an AMPK agonist is used to promote a circadian cycle associated with increased CLOCK/BMAL1 transcriptional activity. In one embodiment the AMPK agonist promotes a sleep effect due to signaling of energy conservation through the corresponding kinase cascade. The method includes administering to a subject an AMPK agonist in an amount sufficient to simulate an energy deficient state in a subject. By “energy deficient state” refers to a state in which the γ subunit of AMPK undergoes a conformation change. Promoting a sleep effect means that such effect is improved in a subject more than would have occurred in the absence of an AMPK agonist.

As described more fully below, the AMPK agonist may be administered orally, parenterally, intramuscularly, intravascularly or by any appropriate route. In one embodiment, the AMPK agonist is administered epidurally. In one embodiment, the AMPK agonist is formulated to promote crossing of the blood-brain barrier.

The disclosure also provide methods of promoting an active state comprising administering an agent that antagonizes an AMPK activity thereby setting the metabolism and activity to a “wake” or “active” cycle. In one embodiment, the AMPK antagonist is an inhibitory antibody. In one embodiment, the AMPK antagonist is a small molecule inhibitors such as Compound C (Dorsomorphin, 6-[4-(2-Piperidin-1-yl-ethoxy)-phenyl)]-3-pyridin-4-yl-pyrrazolo[1,5-a]-pyrimidine), analog, derivative or salt thereof.

The disclosed methods envision the use of any method of administration, dosage, and/or formulation of an AMPK agonist alone or in combination with other circadian regulating agents or sleep aids that have the desired outcome of inducing a desired state of the circadian cycle in a subject receiving the formulation, including, without limitation, methods of administration, dosages, and formulations well known to those of ordinary skill in the pharmaceutical arts.

AMPK agonist of the disclosure may be administered in the form of a drug to a human or an animal. Alternatively, the AMPK agonist may be incorporated into a variety of foods and beverages or pet foods so as to be consumed by humans or animals. The AMPK agonist may be applied to a common food or beverage; or may be applied to a functional food or beverage, a food for a subject suffering a disease, or a food for specified health use, the food (or beverage) bearing a label thereon indicating that it has a physiological function; for example, sleep aid.

The AMPK agonist alone or in combination with other sleep aid or active ingredients may be formulated into a drug product; for example, a peroral solid product such as a tablet or a granule, or a peroral liquid product such as a solution or a syrup.

Modes of administering an AMPK agonist or a formulation in the disclosed method include, but are not limited to, intrathecal, intradermal, intramuscular, intraperitoneal (ip), intravenous (iv), subcutaneous, intranasal, epidural, intradural, intracranial, intraventricular, and oral routes. In a specific example, the AMPK agonist is administered orally. Other convenient routes for administration of an AMPK agonist include for example, infusion or bolus injection, topical, absorption through epithelial or mucocutaneous linings (for example, oral mucosa, rectal and intestinal mucosa, and the like) ophthalmic, nasal, and transdermal. Administration can be systemic or local. Pulmonary administration also can be employed (for example, by an inhaler or nebulizer), for instance using a formulation containing an aerosolizing agent.

In some embodiments, it may be desirable to administer an AMPK agonist or an AMPK agonist locally. This may be achieved by, for example, local or regional infusion or perfusion, topical application (for example, wound dressing), injection, catheter, suppository, or implant (for example, implants formed from porous, non-porous, or gelatinous materials, including membranes, such as sialastic membranes or fibers), and the like.

In other embodiments, a pump (such as a transplanted minipump) may be used to deliver an AMPK agonist or a formulation (see, e.g., Langer Science 249, 1527, 1990; Sefton Crit. Rev. Biomed. Eng. 14, 201, 1987; Buchwald et al., Surgery 88, 507, 1980; Saudek et al., N. Engl. J. Med. 321, 574, 1989). In another embodiment, an AMPK agonist or a formulation is delivered in a vesicle, in particular liposomes (see, e.g., Langer, Science 249, 1527, 1990; Treat et al., in Liposomes in the Therapy of Infectious Disease and Cancer, Lopez-Berestein and Fidler (eds.), Liss, N.Y., pp. 353-365, 1989).

In yet another method embodiment, an AMPK agonist can be delivered in a controlled-release formulation. Controlled-release systems, such as those discussed in the review by Langer (Science 249, 1527 1990), are known. Similarly, polymeric materials useful in controlled-released formulations are known (see, e.g., Ranger et al., Macromol. ScL Rev. Macromol. Chem. 23, 61, 1983; Levy et al., Science 228, 190, 1985; During et al., Ann. Neurol. 25, 351, 1989; Howard et al., J. Neurosurg. 71, 105, 1989). For example, an agonists may be coupled to a class of biodegradable polymers useful in achieving controlled release of a compound, including polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The disclosed methods contemplate the use of any dosage form of an AMPK agonist or formulation thereof that delivers the agonist(s) and achieves a desired result. Dosage forms are commonly known and are taught in a variety of textbooks, including for example, Allen et al., Ansel's Pharmaceutical Dosage Forms and Drug Delivery Systems, Eighth Edition, Philadelphia, Pa.:Lippincott Williams & Wilkins, 2005, 738 pages. Dosage forms for use in a disclosed method include, without limitation, solid dosage forms and solid modified-release drug delivery systems (e.g., powders and granules, capsules, and/or tablets); semi-solid dosage forms and transdermal systems (e.g., ointments, creams, and/or gels); transdermal drug delivery systems; pharmaceutical inserts (e.g., suppositories and/or inserts); liquid dosage forms (e.g., solutions and disperse systems); and/or sterile dosage forms and delivery systems (e.g., parenterals, and/or biologies). Particular exemplary dosage forms include aerosol (including metered dose, powder, solution, and/or without propellants); beads; capsule (including conventional, controlled delivery, controlled release, enteric coated, and/or sustained release); caplet; concentrate; cream; crystals; disc (including sustained release); drops; elixir; emulsion; foam; gel (including jelly and/or controlled release); globules; granules; gum; implant; inhalation; injection; insert (including extended release); liposomal; liquid (including controlled release); lotion; lozenge; metered dose (e.g., pump); mist; mouthwash; nebulization solution; ocular system; oil; ointment; ovules; powder (including packet, effervescent, powder for suspension, powder for suspension sustained release, and/or powder for solution); pellet; paste; solution (including long acting and/or reconstituted); strip; suppository (including sustained release); suspension (including lente, ultre lente, reconstituted); syrup (including sustained release); tablet (including chewable, sublingual, sustained release, controlled release, delayed action, delayed release, enteric coated, effervescent, film coated, rapid dissolving, slow release); transdermal system; tincture; and/or wafer. Typically, a dosage form is a formulation of an effective amount (such as a therapeutically effective amount) of at least one active pharmaceutical ingredient including an AMPK agonist with pharmaceutically acceptable excipients and/or other components (such as one or more other active ingredients). An aim of a drug formulation is to provide proper administration of an active ingredient (such as an AMPK agonist or AMPK antagonist) to a subject. A formulation should suit the mode of administration. The term “pharmaceutically acceptable” means approved by a regulatory agency of the federal or a state government or listed in the U.S. Pharmacopoeia or other generally recognized pharmacopoeia for use in animals, and, more particularly, in humans. Excipients for use in exemplary formulations include, for instance, one or more of the following: binders, fillers, disintegrants, lubricants, coatings, sweeteners, flavors, colorings, preservatives, diluents, adjuvants, and/or vehicles. In some instances, excipients collectively may constitute about 5%-95% of the total weight (and/or volume) of a particular dosage form.

Pharmaceutical excipients can be, for instance, sterile liquids, such as water and/or oils, including those of petroleum, animal, vegetable, or synthetic origin, such as peanut oil, soybean oil, mineral oil, sesame oil, and the like. Water is an exemplary carrier when a formulation is administered intravenously. Saline solutions, blood plasma medium, aqueous dextrose, and glycerol solutions can also be employed as liquid carriers, particularly for injectable solutions. Oral formulations can include, without limitation, pharmaceutical grades of mannitol, lactose, starch, magnesium stearate, sodium saccharine, cellulose, magnesium carbonate, and the like. A more complete explanation of parenteral pharmaceutical excipients can be found in Remington, The Science and Practice of Pharmacy, 19th Edition, Philadelphia, Pa.:Lippincott Williams & Wilkins, 1995, Chapter 95. Excipients may also include, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like. Other examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A formulation, if desired, can also contain minor amounts of wetting or emulsifying agents, or pH buffering agents.

In some embodiments involving oral administration, oral dosages of an AMPK agonist will generally range between about 0.001 mg per kg of body weight per day (mg/kg/day) to about 100 mg/kg/day, and such as about 0.01-10 mg/kg/day (unless specified otherwise, amounts of active ingredients are on the basis of a neutral molecule, which may be a free acid or free base). For example, an 80 kg subject would receive between about 0.08 mg/day and 8 g/day, such as between about 0.8 mg/day and 800 mg/day. A suitably prepared medicament for once a day administration would thus contain between 0.08 mg and 8 g, such as between 0.8 mg and 800 mg. In some instance, formulation comprising an AMPK agonist or antagonist may be administered in divided doses of two, three, or four times daily. For administration twice a day, a suitably prepared medicament as described above would contain between 0.04 mg and 4 g, such as between 0.4 mg and 400 mg. Dosages outside of the aforementioned ranges may be necessary in some cases. Examples of daily dosages that may be given in the range of 0.08 mg to 8 g per day include 0.1 mg, 0.5 mg, 1 mg, 2.5 mg, 5 mg, 10 mg, 25 mg, 50 mg, 100 mg, 200 mg, 300 mg, 400 mg, 500 mg, 600 mg, 800 mg, 1 g, 2 g, 4 g and 8 g. These amounts can be divided into smaller doses if administered more than once per day (e.g., one-half the amount in each administration if the drug is taken twice daily).

For some method embodiments involving administration by injection (e.g., intravenously or subcutaneous injection), a subject would receive an injected amount that would deliver the active ingredient in approximately the quantities described above. The quantities may be adjusted to account for differences in delivery efficiency that result from injected drug forms bypassing the digestive system. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, such as for example 0.1 mg/ml, 0.3 mg/ml, or 0.6 mg/ml, and administered in amounts per day equivalent to the amounts per day stated above. For example, an 80 kg subject, receiving 8 ml twice a day of an intravenous formulation having a concentration of active ingredient of 0.5 mg/ml, receives 8 mg of active ingredient per day.

In other method embodiments, an AMPK agonist or antagonist (or a formulation thereof) can be administered at about the same dose throughout a treatment period, in an escalating dose regimen, or in a loading-dose regime (for example, in which the loading dose is about two to five times a maintenance dose). In some embodiments, the dose is varied during the course of usage based on the condition of the subject receiving the composition, the apparent response to the composition, and/or other factors as judged by one of ordinary skill in the art. In some embodiments long-term administration of an AMPK agonist or antagonist is contemplated, for instance to manage chronic insomnia or sleep-wake cycle disorders.

The disclosure also provides methods of screening for agents that modulate circadian rhythm by measuring AMPK activation or inhibition. The methods of the disclosure for screening for a compound that modulates circadian rhythm involve providing a cell, tissue or subject (e.g., an animal) comprising and AMPK pathway; contacting the subject with an agent suspected of having circadian rhythm modulating activity and measuring the effect on AMPK activity either directly or via downstream kinase activity. The test agent can be provided to a cell preparation, tissue, organ, organism or animal that has at least one observable index of circadian rhythm function and expresses an AMPK. The ability of the agent to modulate circadian rhythm can be tested in a variety of animal species that exhibit indicia of circadian rhythm function, as well as organs, tissues, and cells obtained from such animals, and cell preparations derived there from. An agent that modulates AMPK activity can then be identified as an agent that has putative circadian rhythm modulating activity.

A variety of in vitro screening methods are useful for identifying a antagonist or agonist to be provided in the methods of the disclosure for identifying a compound that modulates circadian rhythm. The ability of a compound to modulate AMPK can be indicated, for example, by the ability of the compound to bind to and activate or inactivate AMPK, block downstream kinase activity, modulate phosphorylation and dephosphorylation (e.g., phosphorylation, dephosphorylation of Cry1 or Cry2), or modulate a predetermined signal produced by AMPK. Therefore, signaling and binding assays can be used to identify an antagonist or agonist of AMPK that is provided in the methods of the disclosure for identifying a compound that modulates circadian rhythm.

An “agent” is any substance or any combination of substances that is useful for achieving an end or result; for example, a substance or combination of substances useful for modulating a protein activity associated with AMPK activation cascade (e.g., AMPK-dependent phosphorylation event), or useful for modifying or affecting a protein-protein interaction or ATP metabolism.

Exemplary agents include, but are not limited to, peptides such as, for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)2 and Fab expression library fragments, and epitope-binding fragments thereof), small organic or inorganic molecules (such as, so-called natural products or members of chemical combinatorial libraries), molecular complexes (such as protein complexes), or nucleic acids.

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc, 114:6568, 1992), nonpeptidal peptidomimetics with glucose scaffolding (Hirschmann et al., J. Am. Chem. Soc, 114:9217-9218, 1992), analogous organic syntheses of small compound libraries (Chen et al., J. Am. Chem. Soc, 116:2661, 1994), oligocarbamates (Cho et al., Science, 261: 1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol, 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produce in a variety of manners including, but not limited to, spatially arrayed multipin peptide synthesis (Geysen, et al., Proc Natl. Acad. Sci., 81(13):3998-4002, 1984), “tea bag” peptide synthesis (Houghten, Proc Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97 (2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one embodiment, high throughput screening methods involve providing a combinatorial chemical or peptide library containing a large number of potential therapeutic compounds (e.g., affectors of AMPK protein-protein interactions). Such combinatorial libraries are then screened in one or more assays as described herein to identify those library members (particularly chemical species or subclasses) that display a desired characteristic activity (such as increasing or decreasing an AMPK protein-protein interaction). The compounds thus identified can serve as conventional “lead compounds” or can themselves be used as potential or actual therapeutics. In some instances, pools of candidate agents may be identify and further screened to determine which individual or subpools of agents in the collective have a desired activity. Agents that affect (e.g., increase or decrease) an AMPK interaction or AMP-dependent phosphorylation of processes may have the effect of modulating circadian rhythms (e.g., sleep behaviour) in a subject and, therefore, are desirable to identify.

In screening methods described here, tissue samples, isolated cells, isolated polypeptides, and/or test agents can be presented in a manner suitable for high-throughput screening; for example, one or a plurality of isolated tissue samples, isolated cells, or isolated polypeptides can be inserted into wells of a microtitre plate, and one or a plurality of test agents can be added to the wells of the microtitre plate. Alternatively, one or a plurality of test agents can be presented in a high-throughput format, such as in wells of microtitre plate (either in solution or adhered to the surface of the plate), and contacted with one or a plurality of isolated tissue samples, isolated cells, and/or isolated polypeptides under conditions that, at least, sustain the tissue sample or isolated cells or a desired polypeptide function and/or structure. Test agents can be added to tissue samples, isolated cells, or isolated polypeptides at any concentration that is not lethal to tissues or cells, or does not have an adverse effect on polypeptide structure and/or function. It is expected that different test agents will have different effective concentrations. Thus, in some methods, it is advantageous to test a range of test agent concentrations.

Methods for detecting protein phosphorylation are conventional (see, e.g., Gloffke, The Scientist, 16(19):52, 2002; Screaton et al., Cell, 119:61-74, 2004) and detection kits are available from a variety of commercial sources (see, e.g., Upstate (Charlottesville, Va., USA), Bio-Rad (Hercules, Calif., USA), Marligen Biosciences, Inc. (Ijamsville, Md., USA), Calbiochem (San Diego, Calif., USA). Briefly, phosphorylated protein can be detected using stains specific for phosphorylated proteins in gels. Alternatively, antibodies specific phosphorylated proteins can be made or commercially obtained. Antibodies specific for phosphorylated proteins can be, among other things, tethered to the beads (including beads having a particular color signature) or used in ELISA or Western blot assays.

In particular methods, the phosphorylation of a polypeptide is increased when such posttranslational modification is detectably measured or when such posttranslational modification is at least 20%, at least 30%, at least 50%, at least 100% or at least 250% higher than control measurements (e.g., in the same test system prior to addition of a test agent, or in a comparable test system in the absence of a test agent, or in a comparable test system in the absence of AMPK).

The amino acid sequences of prototypical AMPK subunits (such as AMPKα1 and/or AMPKα2) (and nucleic acids sequences encoding prototypical AMPK subunits (such as AMPKα1 and/or AMPKα2)) are well known. Exemplary AMPKα1 amino acid sequences and the corresponding nucleic acid sequences are described, for instance, in GenBank Accession Nos. NM_{—}206907.3 (GI:94557298) (Homo sapiens transcript variant 2 REFSEQ including amino acid and nucleic acid sequences); NM_{—}006251.5 (GI:94557300) (Homo sapiens transcript variant 1 REFSEQ including amino acid and nucleic acid sequences); NM_{—}001013367.3 (GI:94681060) (Mus musculus REFSEQ including amino acid and nucleic acid sequences); NMJ)01039603.1 (GI:88853844) (Gallus gallus REFSEQ including amino acid and nucleic acid sequences); and NM_{—}019142.1 (GI: 11862979XRaJfWS norvegicus REFSEQ including amino acid and nucleic acid sequences). Exemplary AMPKα2 amino acid sequences and the corresponding nucleic acid sequences are described, for instance, in GenBank Accession Nos. NM_{—}006252.2 (GI:46877067) (Homo sapiens REFSEQ including amino acid and nucleic acid sequences); NM_{—}178143.1 (GI:54792085)(Mus musculus REFSEQ including amino acid and nucleic acid sequences); NM_{—}001039605.1 (GI:88853850)(Gallus gallus REFSEQ including amino acid and nucleic acid sequences); and NM_{—}214266.1 (GI:47523597)(Mus musculus REFSEQ including amino acid and nucleic acid sequences).

In some method embodiments, a homolog or functional variant of an AMPK subunit shares at least 60% amino acid sequence identity with a prototypical AMPKα1 and/or AMPKα2 polypeptide; for example, at least 75%, at least 80%, at least 85%, at least 90%, at least 95%, or at least 98% amino acid sequence identity with an amino acid sequence as set forth in the GenBank Accession Nos. NM_{—}206907.3; NM_{—}006251.5; NMJ)01013367.3; NM_{—}001039603.1; NM_{—}019142.1; NM_{—}006252.2; NM_{—}178143.1; NM_{—}001039605.1; or NM_{—}214266.1. In other method embodiments, a homolog or functional variant of an AMPK subunit has one or more conservative amino acid substitutions as compared to a prototypical AMPKα1 and/or AMPKα2 polypeptide; for example, no more than 3, 5, 10, 15, 20, 25, 30, 40, or 50 conservative amino acid changes compared to an amino acid sequence as set forth in as set forth in GenBank Accession Nos. NM_{—}206907.3; NM_{—}006251.5; NM_{—}001013367.3; NM_{—}001039603.1; NM_{—}019142.1; NM_{—}006252.2; NM_{—}178143.1; NM_{—}001039605.1; or NM_{—}214266.1. Exemplary conservative amino acid substitutions have been previously described herein.

Some method embodiments involve a functional fragment of AMPK or a subunit thereof (such as AMPKα1 and/or AMPKα2). Functional fragments of AMPK or a subunit thereof (such as AMPKα1 and/or AMPKα2) can be any portion of a full-length or intact AMPK polypeptide complex or subunit thereof (such as AMPKα1 and/or AMPKα2), including, e.g., about 20, about 30, about 40, about 50, about 75, about 100, about 150 or about 200 contiguous amino acid residues of same; provided that the fragment retains at least one AMPK (or AMPKα1 and/or AMPKα2) function of interest Protein-protein interactions between polypeptides in an AMPK pathway are believed to involve, at least, an AMPKα subunit (such as AMPKα1 and/or AMPKα2).

An “isolated” biological component (such as a polynucleotide, polypeptide, or cell) has been purified away from other biological components in a mixed sample (such as a cell or tissue extract). For example, an “isolated” polypeptide or polynucleotide is a polypeptide or polynucleotide that has been separated from the other components of a cell in which the polypeptide or polynucleotide was present (such as an expression host cell for a recombinant polypeptide or polynucleotide).

The term “purified” refers to the removal of one or more extraneous components from a sample. For example, where recombinant polypeptides are expressed in host cells, the polypeptides are purified by, for example, the removal of host cell proteins thereby increasing the percent of recombinant polypeptides in the sample. Similarly, where a recombinant polynucleotide is present in host cells, the polynucleotide is purified by, for example, the removal of host cell polynucleotides thereby increasing the percent of recombinant polynucleotide in the sample.

Isolated polypeptides or nucleic acid molecules, typically, comprise at least 50%, at least 60%, at least 70%, at least 80%, at least 90%, at least 95% or even over 99% (w/w or w/v) of a sample.

Polypeptides and nucleic acid molecules are isolated by methods commonly known in the art and as described herein. Purity of polypeptides or nucleic acid molecules may be determined by a number of well-known methods, such as polyacrylamide gel electrophoresis for polypeptides, or agarose gel electrophoresis for nucleic acid molecules.

The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. ScL USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., (1999), FEMS Microbiol. Lett., 174:247-250, 1999. Altschul et al. present a detailed consideration of sequence alignment methods and homology calculations (J. Mol. Biol. 215:403-410, 1990). The National Center for Biotechnology Information (NCBI) Basic Local Alignment Search Tool (BLAST™, Altschul et al., J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method.

Specific binding refers to the particular interaction between one binding partner (such as a binding agent) and another binding partner (such as a target). Such interaction is mediated by one or, typically, more noncovalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a protein-protein interaction) can be competitively removed (or displaced) from such association (e.g., protein complex) by excess amounts of either specific binding partner. Such competition assays (or displacement assays) are very well known in the art.

The disclosure also provides methods for identifying agents and agents useful for effecting circadian rhythms and sleep behaviour.

### EXAMPLES

The following examples are provided to illustrate certain particular features and/or embodiments. These examples should not be construed to limit the invention to the particular features or embodiments described.

### Example 1

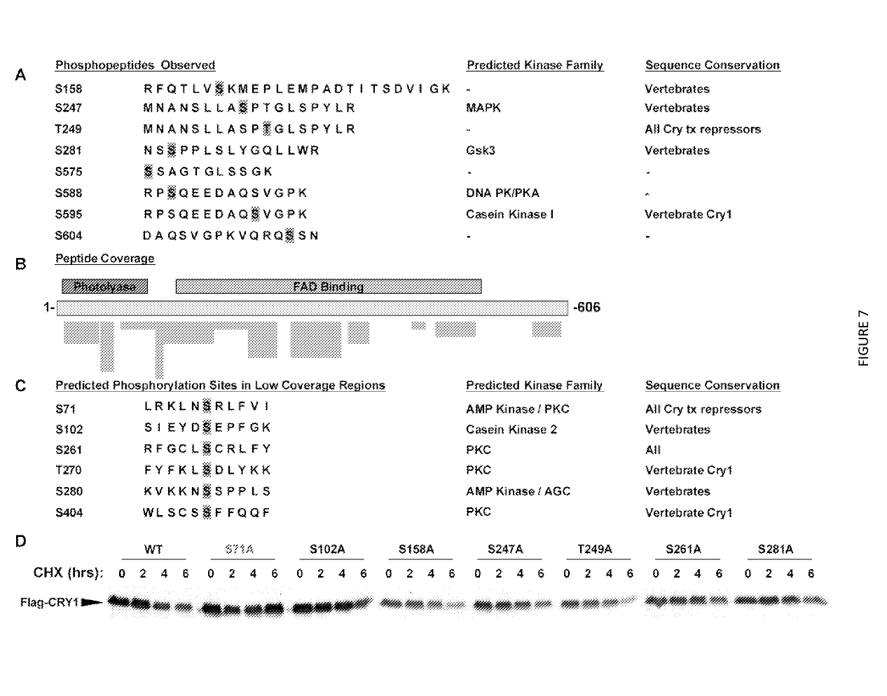

Phosphorylation of CRY1-S71 or -S280 Increases CRY1:FBXL3 Interaction. To explore the role of posttranslational modifications as a mechanism in resetting peripheral clocks, a combination of mass spectrometry and bioinformatics analysis was used to identify eight serine or threonine residues in mCRY1 and mCRY2 that were predicted to be sites of regulated phosphorylation. Non-phosphorylatable mutants were generated for each and found that mutation of serine 71 to alanine stabilized mCRY1, while the remaining mutations had less or no effect on stability (FIG. 7).

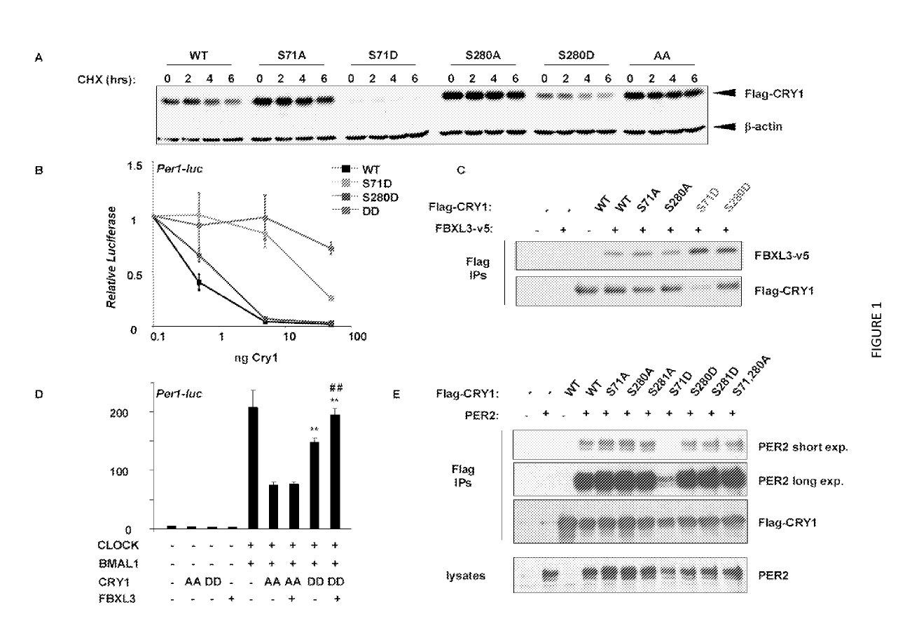

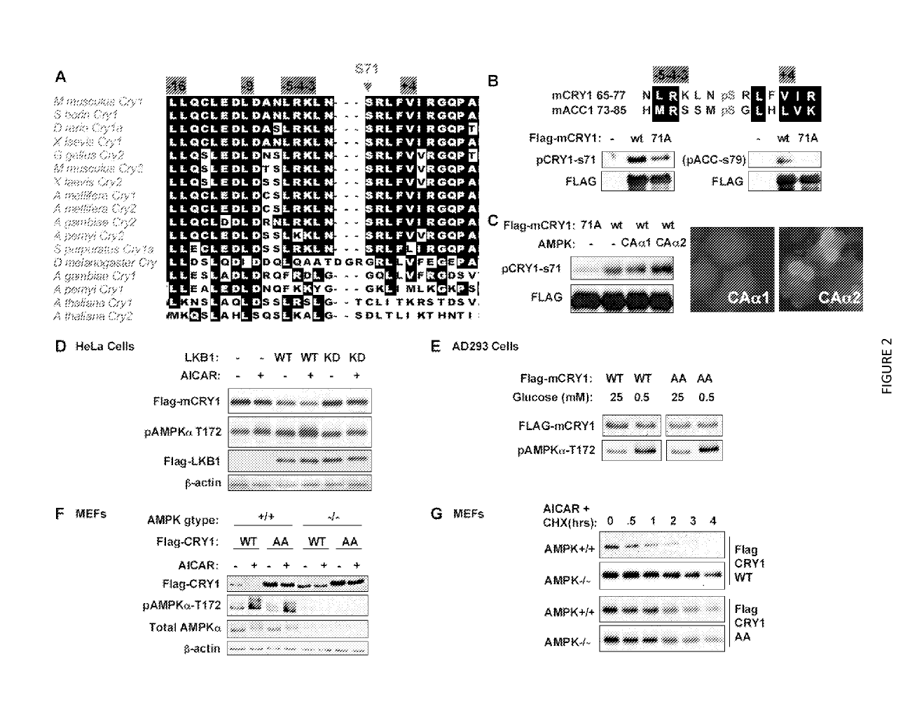

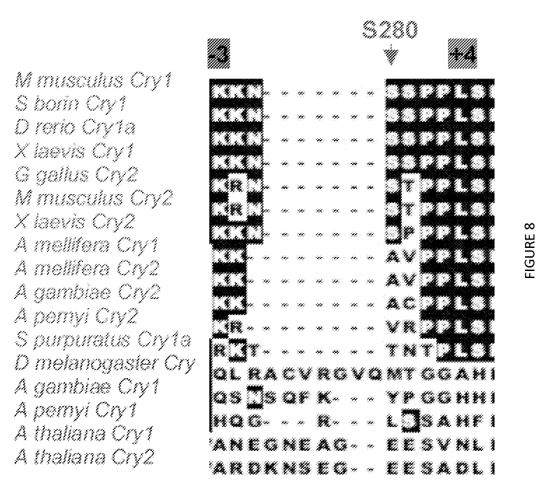

The CRY1-stabilizing mutation affecting serine 71 is particularly intriguing because it conforms well to the optimal sequence phosphorylated by AMPK. Mammalian cryptochromes contain another serine, at position 280 of mCRY1, which also conforms well to the AMPK substrate motif (FIG. 8). The mutation of either serine 71 or serine 280 to a non-phosphorylatable amino acid (alanine) was sufficient to stabilize mCRY1 while mutation of either serine 71 or serine 280 to a phospho-mimetic amino acid (aspartate) was sufficient to destabilize mCRY1 and that mutation of both residues together increased the effects on stability (FIG. 1). In both cases, mutation of serine 71, which is evolutionarily conserved in all non-light sensitive insect cryptochromes and higher organisms (FIG. 2), had a stronger effect than mutation of S280 (FIG. 1A). mCRY1 harboring phospho-mimetic mutations of both S71 and S280 to aspartic acid was undetectable by immunoblot. Consistent with decreased stability of mCRY1 that is phosphorylated at serine 71 and/or serine 280, the phospho-mimetic mutants of those sites were also less effective repressors of CLOCK:BMAL1 transcriptional activity (FIG. 1B). Cryptochromes were originally identified as blue light photoreceptors in plants and later recognized as components of animal circadian clocks. Many insects express one of each type of cryptochrome: a blue light photoreceptor that is degraded upon light exposure (“type 1”) and a transcriptional repressor that participates in circadian transcriptional regulation but is not sensitive to light-induced destabilization (“type 2”). Insect “type 2” cryptochrome proteins, like their mammalian counterparts, oscillate over the course of the day, indicating that their stability must be regulated by a non-light signal, possibly using a conserved mechanism involving FBXL3, an ortholog of which is present in insects (GenBank reference # XM_{—}001120533.1).

To determine whether the instability of the mCRY1 mutants harboring mutations mimicking phosphorylation of serines 71 and 280 reflects increased interaction with FBXL3, Flag-tagged wild type or mutant mCRY1 with v5-tagged FBXL3 were expressed and their binding affinity analyzed by immunoprecipitation of the Flag-tagged mCRY1 followed by immunoblot of the v5-tagged FBXL3. Mutation of either S71 or S280 to aspartic acid increased the binding affinity of mCRY1 for FBXL3 (FIG. 1C), suggesting that phosphorylation of these sites mediates increased interaction between mCRY1 and FBXL3. The double mutant was too unstable to determine its interaction with FBXL3 biochemically.

When these mutants were tested for the ability to repress the transcriptional activity of cotransfected CLOCK and BMAL1, the CRY1 non-phosphorylatable AA mutant proved to be an effective repressor and this repression was not altered by cotransfection of FBXL3. As expected, the double phosphomimetic CRY1 DD was a less effective inhibitor of CLOCK:BMAL1 activity, which may reflect the lower stability of this mutant. In contrast to the lack of FBXL3-mediated effect on CRY1 AA repression, the weak ability of the CRY1 DD to repress CLOCK:BMAL1-driven transcription was lost by co-expression of FBXL3 (FIG. 1D).

The effect of S71, S280 and S281 mutations were examined on the interaction of mCRY1 with its known binding partner PER2. The phosphomimetic mutation of serine 71 (S71D) blocked the interaction between CRY1 and PER2, while the S280D mutant retained PER2 binding, as did the other mutants examined (FIG. 1E). This difference may contribute to the enhanced degradation of CRY1 S71D over CRY1 S280D. Thus, the S71D mutant exhibits decreased binding to PER2 and increased binding to FBXL3, each of which is expected to destabilize CRY1 and which together likely account for the observed instability of CRY1 S71D.

AMPK Mediates Phosphorylation-Dependent Cryptochrome Degradation. The sequence context surrounding serine 71 of mCRY1 suggests that it is an excellent candidate for phosphorylation by AMPK, including not only the nearby preferred sequence specificity (positively charged residues at positions +4 and +3 and hydrophobic residues at positions +5 and +4 relative to the target serine) but even the distal preferred leucine residues at positions −16 and −9 relative to the target serine (FIG. 2A). The amino acid sequence context surrounding 5280 is also suggestive of AMPK phosphorylation according to the proximal preferred sequence specificity (FIG. 8).

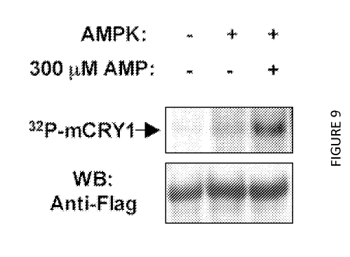

A phospho-specific antibody was generated against a peptide antigen containing phospho-serine surrounded by the sequence context of mCRY1 S71 and observed phosphorylation of exogenously expressed wild type mCRY1 but not the non-phosphorylatable S71A mutant with this antibody (FIG. 2B). The sequence surrounding serine 71 of mCRY1 is similar to that surrounding serine 79 of acetyl coenzyme A carboxylase 1 (ACC1), which is among the best-studied substrates of AMPK (FIG. 2B). Indeed, the antibody raised against a peptide corresponding to residues 73-85 of ACC1 phosphorylated on serine 79 is able to detect wild type mCRY1 but not mCRY1 harboring a mutation that replaces serine 71 with alanine (FIG. 2B), providing additional evidence that serine 71 of mCRY1 can be phosphorylated in vivo and further suggesting that this phosphorylation event may be mediated by AMPK. When a constitutively active mutant of the AMPKα2 catalytic domain (CAa2) was expressed with mCRY1, an increase in phosphorylation of serine 71 (FIG. 2C) was observed, confirming that AMPK can phosphorylate CRY1 in vivo on serine 71. The constitutively active mutant of AMPKα1 (CAa1) was excluded from the nucleus and did not appreciably increase the phosphorylation of serine 71 (FIG. 2C). AMPK was also able to directly phosphorylate mCRY1 in an in vitro kinase assay using purified components (FIG. 9).

Activation of Endogenous AMPK Destabilizes Cryptochromes. Several complementary strategies were used to analyze the contribution of endogenous AMPK to phosphorylation of S71 and 5280 and destabilization of mCRY1. HeLa cells have reduced activation of endogenous AMPK in response to energy stress due to methylation of the promoter for the AMPK-activating kinase LKB1. Introduction of wild type (WT) but not inactive (KD) LKB1 reduced the levels of exogenously expressed mCRY1 and this reduction was enhanced by adding the AMPK-activating AMP mimetic AICAR in the presence of WT LKB1 but not the KD mutant (FIG. 2D). Similarly, activation of AMPK in AD293 cells by glucose deprivation reduced the expression of transfected wild type mCRY1 (WT) but not a mutant mCRY1 (AA) lacking the predicted AMPK phosphorylation sites (FIG. 2E).

To further examine the role of AMPK in regulating cryptochrome stability, mouse embryonic fibroblasts (MEFs) that are genetically wild type (WT) or null (ampka1^{-/-};ampka2^{-/-}) for the catalytic subunits of AMPK (AMPK^{-/-}) were used. Using retroviruses to stably express flag-tagged wild type (WT) or doubly non-phosphorylatable (AA) mCRY1 in these cells, wild type but not AA CRY1 was shown to be acutely degraded upon treatment with the AMPK agonist AICAR only in the wild type cells. In the absence of functional AMPK, AICAR had no effect on either WT or AA CRY1 (FIG. 2F). The regulation of CRY1 stability via AMPK phosphorylation of S71 and S280 was further confirmed by subjecting these cells to a 4-hour time course of cycloheximide treatment in the presence of AMPK-activating AICAR (FIG. 2G). AICAR treatment resulted in reduced stability of wild type but not the non-phosphorylatable mutant of CRY1.

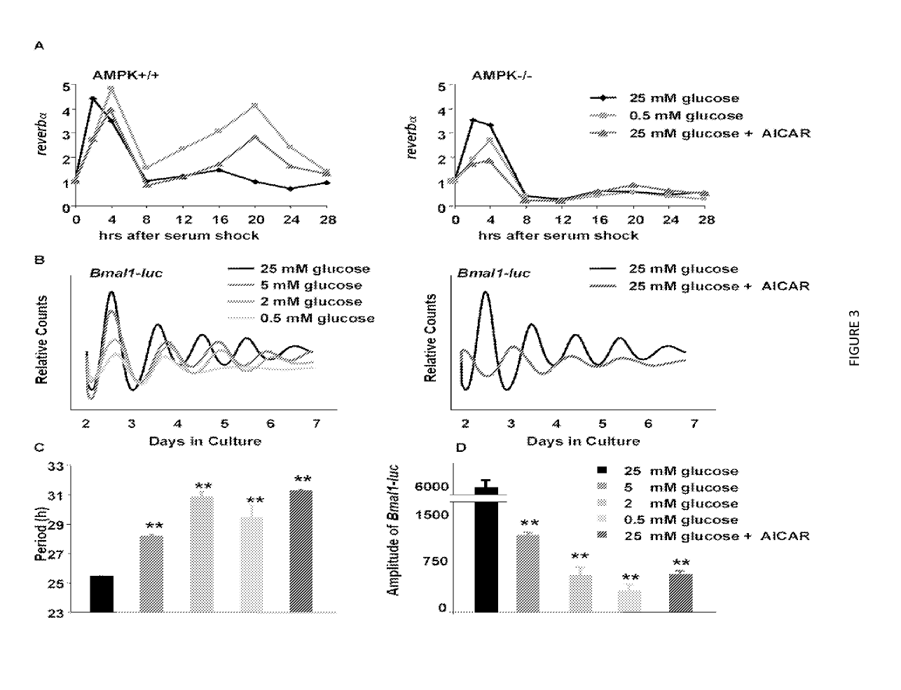

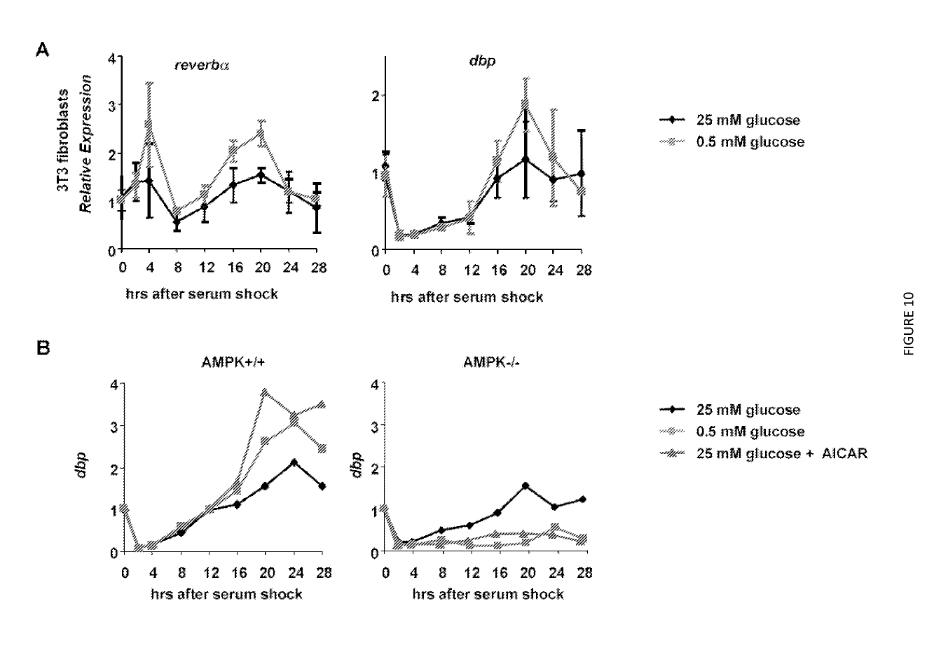

AMPK Contributes to Metabolic Alteration of Circadian Rhythms in Fibroblasts. Given the importance of feeding-derived signals for circadian clock resetting, the regulation of AMPK by glucose availability, and the accumulating evidence of a role for AMPK in cryptochrome destabilization, the effects of AMPK expression and glucose availability were examined on circadian rhythmicity in fibroblasts. When wild type fibroblasts were cultured in medium containing limiting glucose, the amplitude of circadian reverba and dbp expression was significantly enhanced (FIG. 3A and FIG. 10), consistent with a model in which glucose deprivation activates AMPK and reduces CRY stability, leading to de-repression of the CLOCK:BMAL1 targets reverba and dbp. As predicted, addition of AICAR to the culture media mimicked the effects of glucose deprivation. Strikingly, neither glucose deprivation nor AICAR treatment affected the expression of reverba and dbp in MEFs lacking AMPK (ampkα^{-/-};ampkα2^{-/-}, “AMPK−/−”) (FIG. 3A and FIG. 10), indicating that the effects of glucose limitation on fibroblast circadian rhythms are mediated by AMPK.

The Bmall promoter is repressed by REVERBa. Therefore, the effects of reducing glucose availability on circadian rhythms was examined using fibroblasts stably expressing luciferase under the control of a Bmall promoter. Under standard (high glucose) culture conditions, high-amplitude circadian rhythms of expression of Bmall-luciferase were observed with a period of 25.3 hours (FIG. 3B, C). Decreasing the amount of glucose in the culture media increased the circadian period up to 30.7 hours. When the Bmall-luciferase expressing cells were cultured in high glucose medium supplemented with AICAR, the circadian period was similar to that observed in low glucose, reinforcing the idea that the circadian effects of glucose deprivation are mediated by AMPK. The increased expression of REVERBα observed under conditions of limited glucose is expected to result in decreased expression of genes that are repressed by REVERBα, including Bmall. Indeed, activation of AMPK, either by decreasing glucose concentration or by AICAR treatment, decreased the amplitude of Bmall-luciferase expression (FIG. 3D). Together, these results indicate that the circadian rhythms of cultured fibroblasts are responsive to alterations in glucose availability and that these effects are mediated by AMPK-directed phosphorylation.

Circadian Regulation of AMPK in vivo. To investigate the diurnal regulation of AMPK, AMPK transcription, localization, and substrate phosphorylation was examined in peripheral organs of intact animals. All experiments were performed using animals maintained in constant darkness following entrainment to a standard light:dark cycle to ensure that the observed effects were circadian rather than diurnal responses to alterations in the external environment.

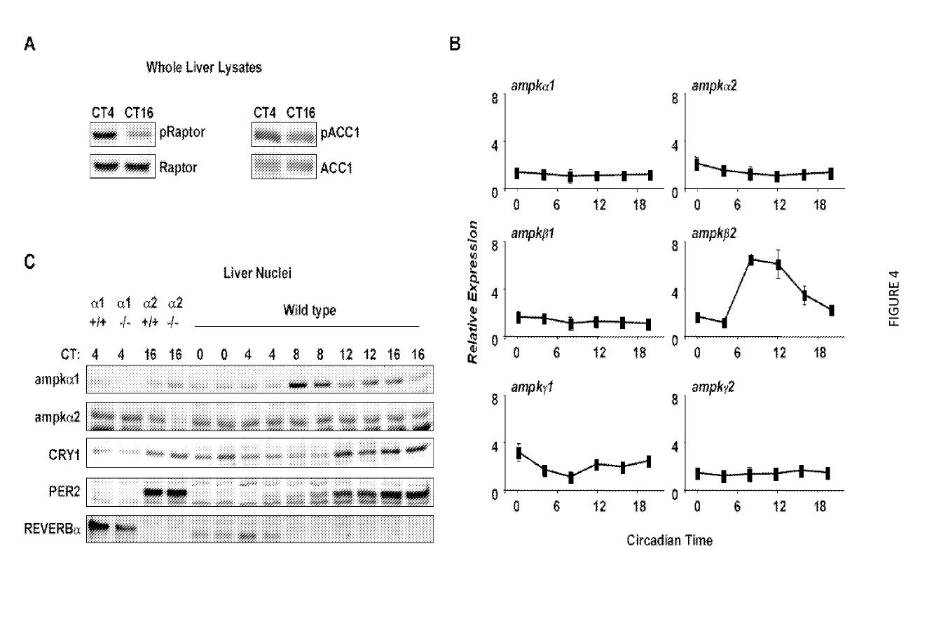

The phosphorylation of both AMPK substrates examined, ACC1-Ser79 and Raptor-Ser792, was reproducibly higher during the subjective day than at night (FIG. 4A), approximately corresponding to the time of day at which negative feedback proteins are unstable, consistent with a model in which rhythmic AMPK activation contributes to the degradation. While exploring the circadian regulation of AMPK in mouse liver, a robust circadian expression of the regulatory ampkβ2 subunit (FIG. 4B), with peak expression concurrent with the time of minimal nuclear cryptochrome proteins (FIG. 4C). AMPKβ2 has been reported to drive the nuclear localization of AMPK complexes, while AMPKβ1-containing complexes are targeted to the plasma membrane. Thus, the circadian transcription of ampkβ2 suggests that oscillating AMPKβ2 diurnally regulates the nuclear localization of AMPKα1 and AMPKα2. To test this hypothesis, the protein levels of AMPKα1 and AMPKα2 in liver nuclei collected across the circadian cycle were measured (FIG. 4C) and observed rhythmicity of nuclear AMPKα1, peaking synchronously with ampkβ2 expression. AMPKα2 contains a nuclear localization signal and was consistently present in the nucleus. The time of peak AMPKα1 nuclear localization is also the time of minimum CRY1 protein in liver nuclei, suggesting that rhythmic nuclear import of AMPK may contribute to the AMPK-mediated phosphorylation and degradation of cryptochromes.

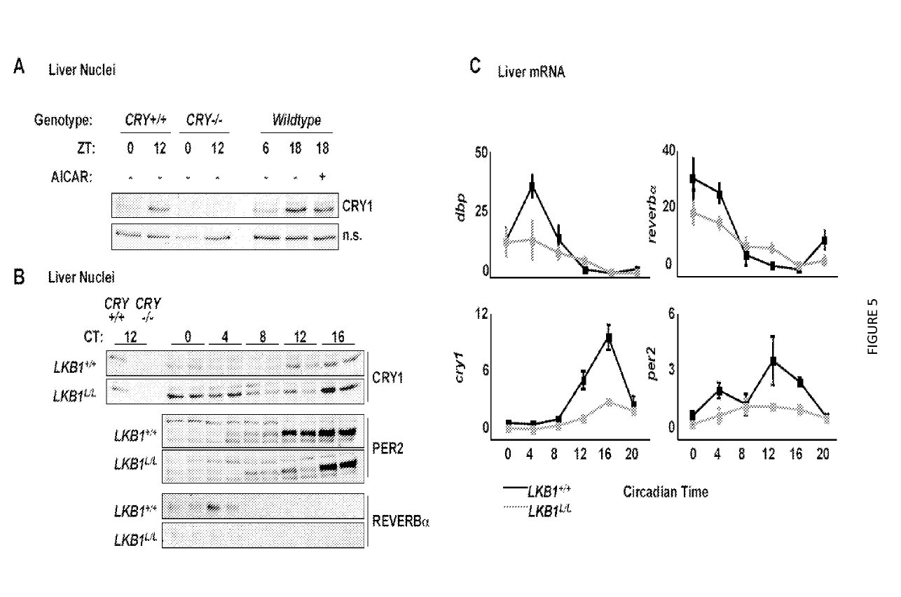

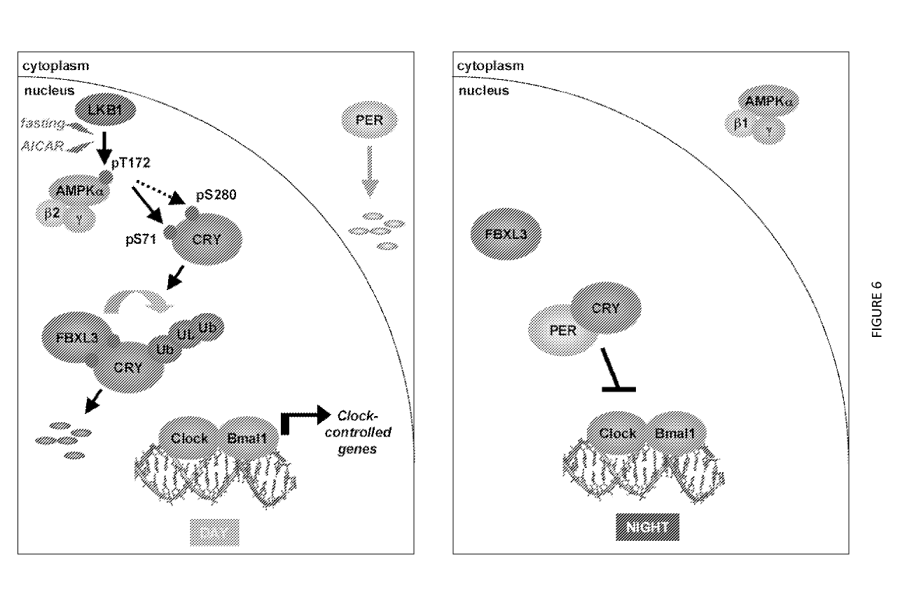

AMPK Alters Circadian Clocks In vivo. Genetic deletion of both AMPKα1 and AMPKα2 in mice leads to early embryonic lethality. Therefore, to further explore the role of AMPK in the liver circadian clock, circadian proteins and transcripts were examined over twenty-four hours in the livers of control mice (LKB1^{+/+}) or littermates harboring loss of lkb1 in hepatocytes (LKB1^{L/L}) housed in constant darkness following entrainment to a light:dark cycle. Liver-specific deletion of lkb1 abolishes AMPK activation in that organ and significantly increased the amount of CRY1 and CRY2 proteins present in liver nuclei across the circadian cycle, particularly during the daytime hours when AMPK was found to be most active in unaltered mice (FIG. 5B). This increase was associated with decreased REVERBα expression (FIG. 5B) in the period corresponding to daylight and decreased amplitude of circadian transcripts throughout the circadian cycle (FIG. 5C). Thus, loss of AMPK signaling in vivo stabilizes cryptochromes and disrupts circadian rhythms, establishing a mechanism of synchronization for light-independent peripheral circadian clocks.

### Materials and Methods

Cells and Cell Culture—AMPK^{+/+} and AMPK^{-/-} mouse embryonic fibroblasts were a gift from Dr. Benoit Viollet. HeLa cells and AD293 cells were purchased from the American Type Culture Collection (ATCC). 3T3 immortalized MEFs were described previously. Unless otherwise indicated, cells were grown in complete Dulbecco's Modified Eagle Medium (DMEM) (Invitrogen cat#11995 or cat #11965) supplemented with 10% fetal bovine serum, penicillin and streptomycin in a 37° C. incubator maintained at 5% CO₂. In experiments in which glucose concentrations were manipulated, cells were grown in minimal DMEM (Sigma cat#D5030) supplemented with glutamine, non-essential amino acids, penicillin, streptomycin and the indicated amounts of D-glucose or glucose-free DMEM (Invitrogen cat#11966) supplemented with penicillin, streptomycin, L-glutamine, and the indicated amounts of D-glucose. Experiments using 0.5 mM glucose were supplemented with D-mannitol to control for osmolar effects. Cell stimulation was performed using complete DMEM with 50% horse serum (Invitrogen cat#26050) and conducted as previously described.

Plasmids and Transfections—pDONR221 and pcDNA3.1/v5-His-TOPO were purchased from Invitrogen; pcDNA3-2xFlag-mCRY1(WT) and pcDNA3-PER2 were gifts from Dr. Charles Weitz; pCMV-SPORT6-Fbx13 was purchased from Open Biosystems and FBXL3 was cloned into pcDNA3.1/v5-His-TOPO by standard protocols; flag-LKB1, myc-AMPKα1 and myc-AMPKα2 constructs were previously described, and the constitutively active alleles (CAa1 and CAa2) were generated by inserting a stop codon after residue T312. All mutations were generated using Stratagene Site-Directed Mutagenesis protocols. Transfections were carried out using FuGene HD (Roche).

Generation of Viruses and Stable Cell Lines—pLXSP3puro expression clones were transfected into AD293 cells along with pCL-Ampho for virus production. Viral supernatants were collected 48 hours after transfection, filtered through a 0.45 μm filter, supplemented with 6 ug/ml polybrene and added to parental cell lines. After 4 hours, additional media was added to dilute the polybrene to <3 ug/ml. 48 hours after viral transduction, the infected cells were split into selection media containing 1-5 ug/ml puromycin. Selection media was replaced every 2-3 days until selection was complete.

Mass Spectrometry—AD293 cells transfected with Flag-mCRY1 were treated with 10 uM MG132 for 6 hours and lysed in buffer containing 1% Tx-100. Flag-mCRY1 was purified on M2-agarose (Sigma) and separated from contaminants by SDS-PAGE; the Coomassie-stained band was excised, rinsed twice in HPLC-grade 50% acetonitrile, and sent to the Beth Israel Deaconess Medical Center Mass Spectrometry facility.

Preparation of Protein Extracts, Immunoprecipitation and Immunoblotting—Whole cell extracts were prepared in Lysis Buffer containing 1% Triton X-100 as previously described and liver nuclear extracts were prepared by the NUN procedure. Antibodies used were anti-Flag M2 agarose, anti-v5 agarose, anti-Flag polyclonal, anti-v5 polyclonal, and anti-βactin from Sigma; CRY11A, CRY21A and PER21A from Alpha Diagnostics International; anti-phosphoACC1(S79), anti-ACC1, anti-phospho-AMPKa, anti-phospho-Raptor, anti-Raptor and anti-REVERBa from Cell Signaling Technologies; anti-AMPKα1 and anti-AMPKα2 from Upstate Biotechology; and a polyclonal antiserum raised against a phosphopeptide containing phospho-CRY1(S71) and surrounding residues generated in collaboration with Millipore.

In vitro Phosphorylation Assay—Flag-mCRY1 was purified from transfected AD293 cells and combined with ³²P-ATP and purified AMPK (from Upstate Biotechnology) in the presence or absence of 300 uM AMP for 30 minutes at room temperature. The reaction mixture was separated by SDS-PAGE and transferred to nitrocellulose. Following radioactive visualization by phosphoimager, the nitrocellulose was immunoblotted for the Flag tag.

Real Time Bioluminescence Monitoring—The human osteosarcoma U2OS reporter cell line stably expressing a Bmall promoter driven luciferase has been described. 2×10⁴ cells were plated in 35-mm dishes and grown to confluency over 3 days in DMEM supplemented with 10% serum. Confluent cells were stimulated with 50% horse serum for 2 hours, then transferred to media containing 0.1% dialyzed serum and varying amounts of glucose as described above. Bioluminescence was continuously recorded by a LumiCycle apparatus from Actimetrics, Inc.

Gene Expression—RNA was extracted from livers or cultured fibroblasts with Trizol or using the Qiagen RNeasy purification system. cDNA was prepared using the SuperscriptII reverse transcriptase (Invitrogen) and analyzed for gene expression using quantitative real-time PCR with either SYBR green (Invitrogen) or TaqMan (Applied Biosystems) chemistry. Primer sequences are available upon request.

Mice—LKB1^{fl/fl} mice were a gift from Dr. Ronald De Pinho, Cry1^{-/-};Cry2^{-/-} mice were a gift from Dr. Aziz Sancar. Adenovirus expressing Cre recombinase was from the University of Iowa Transgenic Core facility. All animal care and treatments were in accordance with the Salk Institute guidelines for the care and use of animals.

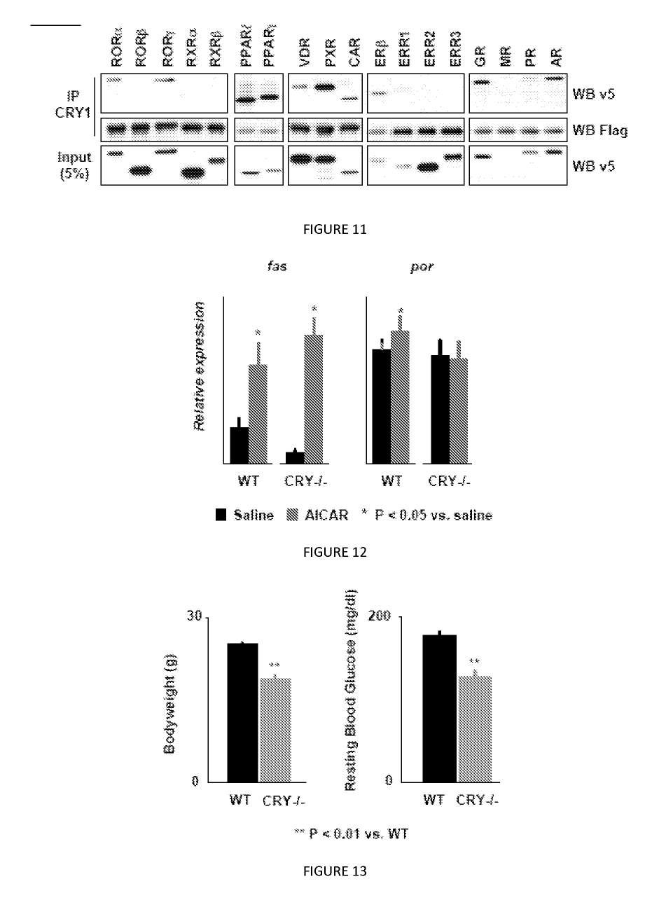

The disclosure demonstrates that mCRY1 indeed interacts with 20 of 47 nuclear hormone receptors that have been examined thus far, and interacts especially well with PPARd (FIG. 11). Furthermore, gene expression in the livers of wildtype and cryptochrome-deficient mice injected with saline or the AMPK-activating drug AICAR demonstrate that cryptochromes are required for AICAR-induced activation of a subset of the genes (FIG. 12). Furthermore, the effect on metabolic physiology of genetic disruption of both Cry1 and Cry2 in mice is examined. The data indicate that Cry1^{-/-};Cry2^{-/-} mice have significantly lower body weight and significantly reduced resting blood glucose than wild type controls (FIG. 13). Collectively, this data suggest that mammalian cryptochromes function as previously unrecognized sensors of cellular energy status, that they play a role in organismal energy homeostasis and that pharmacological modulation of cryptochromes may be useful in the treatment of metabolic disorders.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

1. A method of identifying an agent for modulating metabolism or circadian rhythms, comprising contacting the agent with a Cry1 and/or Cry2 protein and measuring the ability of the agent to phosphorylate or dephosphorylate the Cry1 and/or Cry2, wherein an agent that phosphorylates or dephosphorylates the Cry1 and/or Cry2 is a small molecule agent useful for modulating metabolism or circadian rhythms.

2. The method of claim 1, wherein the agent affects phosphorylation at S71 and/or 5280 of Cry1 and/or Cry2.

3. The method of claim 1, further comprising measuring changes in activity of AMPK.

4. The method of claim 1, wherein the agent decreases stability of Cry1 and/or Cry2.

5. The method of claim 4, wherein the agent promotes a rest state.

6. The method of claim 1, wherein the agent is selected from the group consisting of a peptide, a polypeptide, an antibody, an antibody fragment, a nucleic acid and a small molecule.

7. The method of claim 1, wherein the agent is an AMPK agonist.

8. A composition comprising an agent identified by the method of claim 1, wherein the agent decreases stability of Cry1 and/or Cry2.

9. A method of treating a metabolic or circadian disease or disorder in a subject, comprising contacting the subject with an effective amount of an agent identified by the method of claim 1, wherein the agent promotes phosphorylation or dephosphorylation of Cry1 and/or Cry2.

10. The method of claim 9, wherein the agent is capable of modulating cryptochrome transcriptional co-regulator function in the subject.

11. The method of claim 9, wherein the agent modulates peroxisome proliferator activated receptors (PPAR) alpha, beta(delta) and gamma.

12. The method of claim 9, wherein the agent is an AMPK agonist selected from the group consisting of biguanide derivatives, AICAR, metformin or derivatives thereof, phenformin or derivatives thereof, leptin, adiponectin, AICAR (5-aminoimidazole-4-carboxamide, ZMP, DRL-16536, BG800 compounds (Betagenon), and furan-2-carboxylic acid derivative.

13. The method of claim 9, wherein the subject is a mammal.

14. The method of claim 9, wherein the effective amount is from about 0.5 mg/kg per day to about 100 mg/kg per day in a single dose or in divided doses.

15. The method of claim 9, wherein the agent is formulated for oral administration, intravenous injection, intramuscular injection, epidural delivery, intracranial, topical, intraocular, suppository or subcutaneous injection.

16. A composition comprising an agent that modulates phosphorylation of CRY1 and/or CRY2 or modulates cryptochrome transcriptional co-regulator function and at least one other circadian rhythm or metabolic modifying agent.

17. The composition of claim 16, wherein the at least one other circadian rhythm modifying agent is a sleep aid.

18. The composition of claim 16, wherein the composition comprises an AMPK agonist selected from the group consisting of biguanide derivatives, AICAR, metformin or derivatives thereof, phenformin or derivatives thereof, leptin, adiponectin, AICAR (5-aminoimidazole-4-carboxamide, ZMP, DRL-16536, BG800 compounds (Betagenon), and furan-2-carboxylic acid derivative.

19. The composition of claim 16, wherein the composition is formulated for oral administration, intravenous injection, intramuscular injection, epidural delivery, topically, by suppository, ocular delivery, intracranial delivery, or subcutaneous injection.

20. A method for modulating sleep in a mammal comprising, administering to the mammal an effective amount of CRY1 or CRY2 destabilizing agent to modulate circadian rhythms or metabolism in the mammal.

21. The method of claim 20, wherein the mammal is a human.

22. The method of claim 20, wherein the circadian rhythm is sleep behavior.

23. A method for identifying an agent that modulates circadian rhythms or sleep in a subject, comprising:
(a) contacting a sample from the subject comprising a AMPK and/or LKB1 pathway with at least one test agent; and
(b) comparing an activity or stability of a CRY1 and/or CRY2 pathway in the presence and absence of the test agent wherein a test agent the changes that activity or stability of CRY1 and/or CRY2 is indicative of an agent that has circadian rhythm modulating activity.

24. A method of identifying an agent that modulates circadian or metabolic cycles in a cell comprising contacting the cell with the agent, wherein the cell comprises an AMPK pathway and/or LKB1 pathway including a Cry1 and/or Cry2 and measuring the effect of the agent on Cry1 and/or Cry2 activity, wherein a change in activity of Cry1 and/or Cry2 is indicative of an agent that can modulate circadian or metabolic cycles.

25. A method of determining a metabolic or circadian rhythm disease or disorder in a subject comprising measuring the stability of CRY1 and/or CRY2 in a tissue from the subject during a 24 hour period, wherein a period of long-term stability of CRY1 and/or CRY2 in the presence of normal or excess ATP concentrations is indicative of a metabolic or circadian rhythm disease or disorder in the subject.

26. A method of promoting rest and fat catabolism in a subject comprising administering an AMPK agonist in a subject during a nocturnal phase of a circadian cycle, wherein the AMPK agonist decreases the stability of CRY1 and/or CRY2 in the subject.
